(19) United States
(12) Patent Application Publication
(10) Pub. No.: US 2021/0219990 A1
(43) Pub. Date: Jul. 22, 2021

(54) TOOL FOR PRODUCING A RECESS IN BONE TISSUE

(71) Applicant: Waldemar Link GmbH & Co. KG, Hamburg (DE)

(72) Inventor: Klaus DMUSCHEWSKY, Hamburg (DE)

(21) Appl. No.: 16/303,678

(22) PCT Filed: May 24, 2017
(86) PCT No.: PCT/EP2017/062584

§ 371 Date: Nov. 21, 2018

(30) Foreign Application Priority Data
May 25, 2016 (EP) 16171293.0

(51) Int. Cl. A61B 17/16

(52) U.S. Cl. CPC A61B 17/164; A61B 17/1631; A61B 17/1668

(57) ABSTRACT

The present invention provides a tool for producing a recess in bone tissue for an implant, said tool comprising a first elongated tool segment with a first longitudinal axis and a receiving area, said receiving area having, about the first longitudinal axis, a first functional surface that is orientated substantially radially outwards and a second functional surface that is orientated radially inwards. The tool furthermore comprises a second elongated tool segment with a second longitudinal axis and an insertion section that can be inserted into the receiving area of the first tool segment, said insertion section having, about the second longitudinal axis, at least one functional surface that is orientated radially outwards and interacts with the first functional surface of the first tool segment that is orientated radially outwards. In addition, one of the at least one functional surfaces of the insertion section interacts with the second functional surface of the first tool segment. One of the interacting functional surface pairs is thereby engaged with one another for transmitting torque and the other of the interacting functional surface pairs allows a relative movement therebetween.

### FIELD OF THE INVENTION

The present invention relates to a tool for preparing an implantation site for an implant and in particular for producing a recess in the bone tissue of a long bone. The present invention furthermore relates to a set consisting of an implant and the tool.

### PRIOR ART

When anchoring an implant in a long bone, the medullary canal present in the diaphysis of the long bone can be used to guide and, under certain circumstances, also to stabilise the implant. This has the advantage that less bone tissue has to be removed in order to produce the opening or recess in the bone for receiving the implant. In general, the aim when anchoring an implant in a long bone is accordingly to insert the implant into the medullary canal in such a manner that the bone material is preserved as far as possible. However, as is described below, this is only possible to a limited extent with the tools and techniques available in the prior art.

Two techniques are in particular used to prepare the bone. One technique is the use of a reamer which is inserted at one end of the long bone and which, by means of rotation about it longitudinal axis, produces a rotationally symmetrical opening corresponding to the reamer in the bone. The bone can thereby be pre-drilled with a drill prior to inserting the reamer. This technique allows a quick and precise generation of the recess required to insert the implant.

A further technique is the use of a hand rasp. In contrast to the reamer, a non-rotationally symmetrical recess can be produced in the bone using such a rasp. The rasp is moved linearly along a path of motion to produce a cutting motion. The cutting motion hereof thus fundamentally differs from the rotary cutting motion of the reamer. More specifically, the advancing motion and the cutting motion coincide in the rasp. In a reamer, the cutting motion is, by contrast, separated into a linear advancing motion and a rotary cutting motion.

As a consequence hereof, it is possible using a rasp to produce a recess in the bone tissue that can accommodate an implant with a curve in the longitudinal direction. Such a curve can be provided in order to adapt the implant to the geometry of the respective bone. Such implants are also referred to as being anatomically adapted.

However, this rasp technique has the disadvantage that it is not possible to achieve the same geometrical accuracy when removing the bone tissue as can be achieved with the aforementioned reamer. The main reason for this is that the cutting and advancing motion is performed by hand along a curved path of motion whereas the rotary cutting motion of the reamer is usually performed via a drive and only the linear direction of advancement is manually guided by hand. As a result, it is easier for the surgeon to guide a reamer since he only has to control the advancing motion whilst the cutting motion is carried out by the rotation of the reamer.

Irrespective thereof, a recess that is as precise as possible must also be produced using a rasp, especially in the case of implants that are anchored with the aid of bone ingrowth, in order to achieve sufficient primary stability by pressing the implant into the bone. So that the recess generated in the bone deviates as little as possible from the intended shape, the cross-section of the rasp generally increases from the leading front side towards the rear to a much greater extent than that of a reamer. As a result hereof, the recess is enlarged over its entire length with each working stroke owing to the movement of the rasp. In other words, due to this design of the rasp, the surgeon does not have to pay as much attention that he is precisely following the already taken motion vector since material is removed over the entire length of the rasp and thus a certain amount of correction is possible with each advancing motion.

In contrast, the cross-section of the recess can be kept constant with a reamer even though the reamer continues to remove bone tissue further forward relative to the direction of advancement. As a result, the design of the implant can be functionally better adapted with respect to the material properties.

Both techniques have the disadvantage that they are not able to intraoperatively react to the individual anatomy of the respective long bone. The result is often unwanted cuts in the bone tissue. This is especially disadvantageous in the case of injuries to the cortical bone tissue in the area of the diaphysis. As a result hereof, the long bone is structurally weakened in this area. In addition, the induced notch effect makes the bone vulnerable to bending stresses and, in the worst case, can lead to bone fracture.

Furthermore, so-called stress shielding can be caused at the distal end of the implant shaft, which is only supposed to be guided but not supported by the medullary cavity, i.e. the implant rests on the cortical tissue surrounding the medullary cavity. An incorrect load is placed on the bone owing to an introduction of force in the area of the diaphysis. The bone tissue responds by building up bone tissue in the area where the unnatural force is introduced and by bone resorption at the site where the force is actually supposed to be introduced. This phenomenon is particularly known for joint endoprostheses and can lead to failure of the bone tissue and the implant or prosthesis.

### SUMMARY OF THE INVENTION

The aim was therefore to provide a tool for opening a long bone for an implant, whereby the tool should be able to respond to the anatomical conditions of a bone, in particular a long bone. At the same time, the resection of bone tissue should be kept as low as possible. In other words, the tool of the present invention should make it possible to prepare an implantation site whilst preserving as much bone as possible and, at the same time, to enable provision with an anatomically adapted yet bone-preserving implant.

In view of this challenge, the present invention provides a tool for producing a recess in bone tissue for an implant, said tool comprising a first elongated tool segment with a first longitudinal axis and a receiving area. About the first longitudinal axis, the receiving area has a first functional surface that is orientated substantially radially outwards and a second functional surface that is orientated radially inwards. The tool furthermore comprises a second elongated tool segment with a second longitudinal axis and an insertion section that can be inserted into the receiving area of the first tool segment, with at least one functional surface that is orientated radially outwards being formed on the insertion section about the second longitudinal axis, which functional surface interacts with the first functional surface of the first tool segment that is orientated radially outwards. In addition, one of the at least one functional surfaces of the insertion section interacts with the second functional surface of the first tool segment. One of the interacting functional surface pairs is thereby engaged with one another for transmitting torque, and the other of the interacting functional surface pairs allows a relative movement therebetween.

Such a two-part tool has the advantage that when generating the predefined recess for a prosthesis, it can adapt to a certain extent to the respective anatomical situation of the patient, as will be described below.

The insertion section is received radially offset to the first longitudinal axis between a functional surface of the receiving area that is orientated radially inwards and a functional surface of the receiving area that is orientated radially outwards. In addition, the longitudinal axes of the two tool segments intersect at an angle. In the state where it is inserted into the receiving area, the functional surface of the second tool segment that is orientated radially outwards can, due to this construction, move about the first functional surface of the first tool segment that is orientated radially outwards without any rotation being transmitted between the longitudinal axes of the two tool segments. This means that if the first or second tool segment is held, one end of the insertion section just like the other end opposite to this end can be moved on a circulating path. In other words, one of the tool segments rotates about the longitudinal axis of the other tool segment.

If, on the other hand, the front tool segment, when viewed in the direction of advancement, is guided and the other tool segment is rotated, it is possible to transmit a rotational movement between the two longitudinal axes via the engaged pair of functional surfaces. The pair of functional surfaces that allows a relative movement is in contact with one another and thus supports the insertion section in the receiving area. In other words, the two functional surfaces slide relative to one another. As a result, the two longitudinal axes that are arranged at an angle to one another remain in the same absolute plane and a rotation and a torque are transmitted from one to the other longitudinal axis. As a consequence hereof, a recess is formed in the bone tissue, which corresponds to the external geometry of the tool and in which a corresponding implant can be anchored.

Owing to these properties of the tool segments, the front tool segment can follow the path of least resistance when inserted into the bone tissue. In other words, the bone tissue influences the path of advancement by means of its density and structure. As a result of this “guiding”, the orientation of the tool may be changed by rotation of the one tool segment about the longitudinal axis of the other tool segment. No or only a partial transmission of the rotational movement and the torque from one longitudinal axis to the other longitudinal axis thereby occurs, whilst another part causes a rotation of the other tool segment about this one longitudinal axis. In particular the cortical bone tissue of the diaphysis of a long bone can be preserved in this manner since the front tool segment aligns itself to a certain extent autonomously along the medullary canal during insertion into this medullary canal, but the basic geometry of the recess for the implant is thereby retained. In other words, the tool preserves the surrounding bone tissue by removing as little bone tissue as possible when creating the recess, or by first removing the bone tissue with a lower density.

As a result, the arrangement of the two tool segments at an angle generates a predefined recess or opening for an implant shaft in the bone, which has a corresponding angle. Thus, the tool according to the invention combines the advantages of the two aforementioned techniques in that the surgeon only has to perform the advancing motion whereas the rotary cutting motion can be effected by a drive, and the implantation of an implant that is more anatomically adapted is at the same time made possible.

In a further embodiment of the tool, the first functional surface of the first tool segment is formed by a projection in the receiving area, the projection preferably being conical or cylindrical.

This design of the first functional surface of the first tool segment in particular facilitates the insertion of the insertion section of the second tool segment.

The first functional surface of the first tool segment is preferably a conical surface, in particular a conical surface with a circular cross-section. The projection preferably tapers in the direction of the receiving area opening. If the projection is a cylindrical projection, then it too preferably has a circular cross-section.

In a particularly preferred embodiment of the tool, the engaged pair of functional surfaces for transmitting torque is engaged with a form fit.

The use of a form fit as compared to a friction fit for the transmission of torque has the advantage of defined kinematics, with which the risk of slippage between the bodies is prevented.

In a further preferred embodiment of the tool, the cross-sections of the functional surfaces of the functional surface pairs have a substantially circular circumference perpendicular to the respective longitudinal axis.

This embodiment leads to the first and second tool segments being arranged at a constant angle relative to one another so that in the receiving area, the insertion section of the second tool segment moves substantially in a plane perpendicular to the longitudinal axis of the first tool segment on a circular path about this longitudinal axis.

Owing to the constant angle, the advancing motion can be carried out by the surgeon, in particular by hand, with precise control. The recess made in the bone tissue as a result hereof thereby corresponds to the tool segments introduced into the bone tissue and thus to the predefined geometry and dimensions intended for the implant.

In this context, a substantially circular circumference is also to be understood as a circumference which can periodically deviate from a perfect circular shape. This is the case, for example, if the respective pair of functional surfaces transmits a torque by means of a form-fit engagement. In order to achieve such a fit, recesses or projections must be provided on the circumference of the functional surfaces with a shape that enables an engagement between the functional surfaces.

In a further particularly preferred embodiment, the first tool segment and the second tool segment comprise at least two separate functional surface pairs interacting therebetween.

Owing to the fact that each pair of functional surfaces respectively comprises two functional surfaces of its own in this embodiment, the tool can be provided with a smaller diameter than a tool in which a functional surface is engaged in one region with a functional surface for transmitting a torque and is in contact with a further functional surface in another region in which a relative movement is performed.

In a further embodiment, the first tool segment and the second tool segment comprise at least three separate functional surface pairs interacting therebetween. The first pair of functional surfaces hereby allows a relative movement therebetween, the second pair of functional surfaces supports the insertion section in the receiving area, and the torque can be transmitted between the first and the second tool segments by the third pair of functional surfaces.

In this embodiment, this functional division, as explained below, has the advantage of a very precise sequence of motion. As described above, the insertion section lies on the one side opposite to a functional surface of the receiving area that is orientated radially inwards and on the other side lies opposite to a functional surface of the receiving area that is orientated radially outwards. The pair of functional surfaces for the transmission of torque and the pair of functional surfaces for support thereby act on the same side, whereas the pair of functional surfaces that allows a relative movement acts on the other side. Since torque transmission and support or bearing occur on the same side, the supporting pair of functional surfaces thus rolls against one another. This also means that the pair of functional surfaces which is in contact with one another and allows a relative movement also has a supporting function.

Furthermore, this embodiment has the advantage that the transmission of torque over the entire circumference between the receiving area and the insertion section is made possible since the second pair of functional surfaces assumes the support function.

In a further embodiment, the functional surface of the first tool segment which is orientated radially outwards is exchangeable and preferably part of a third tool segment.

In this embodiment, the adaptability of the tool to the anatomy of a patient is increased since the angle between the longitudinal axes of the first and second tool segments can be changed by exchanging the first functional surface. This property of the first tool segment of this embodiment advantageously complements the exchangeability of the second tool segment, which, as stated above, can be inserted into the receiving area of the first tool segment via the insertion section.

If the exchangeable functional surface is part of a third tool segment, this makes it possible to additionally work and adapt the recess in the bone tissue in its proximal region.

Furthermore, the tool can be extended in this manner as of a predetermined depth of the recess. As a result, the length of the tool is adjustable such that it can be guided in a simple manner almost irrespective of the depth of the recess.

In a further embodiment, at least one of the tool segments comprises at least one cutting element on an outward facing circumferential surface.

Such a provided cutting element is particularly advantageous if a larger volume of bone tissue is to be removed or if the bone tissue is very dense tissue. The latter is the case, for example, with very dense cancellous bone and cortical bone, such that an adaptation of the recess in the bone tissue to a geometry intended for the implant cannot be achieved by compacting the bone tissue.

In a particularly preferred embodiment, the receiving area of the first tool segment comprises a holding surface which is formed by an internal cross-sectional extension of the receiving area and on which a corresponding cross-sectional extension of the insertion section is supported in the inserted state such that the second tool segment is held in the first tool segment.

In this embodiment, the second tool segment is held in the first tool segment and is secured to the extent that after insertion of the insertion section into the receiving area of the first tool segment, the tool is held via the annular support surface until it is placed on the bone tissue.

Owing to the cross-sectional extension in the receiving area of the first tool segment, an undercut is created which forms a step that extends substantially perpendicular to the longitudinal axis of the first tool segment. A shoulder can be supported hereon, which is formed by the corresponding cross-sectional extension on the insertion section of the second tool segment and which faces away from the tip of the insertion section.

It is also possible to provide the holding surface of the first tool segment as a circumferential groove in the receiving area, in which a corresponding projection of the insertion section engages. A simple falling out of the second tool segment from the first tool segment is in any case prevented.

In a further, particularly preferred embodiment, a pair of functional surfaces is arranged at the height of the intersection of the first longitudinal axis and the second longitudinal axis, said pair comprising a functional surface of the insertion section that is orientated radially outwards and a functional surface of the receiving area that is orientated radially inwards, said pair of functional surfaces being engaged preferably for torque transmission.

The arrangement of the functional surfaces at the intersection of the two longitudinal axes leads to a particularly precise and simple guidance of the second tool segment in the first tool segment. In this embodiment, the two functional surfaces can in particular be in contact over their circumference, and thus these functional surfaces are sufficient for specifically supporting the tool segments with respect to their rotational degree of freedom. This applies both to a pair of functional surfaces for torque transmission as well as to a pair of functional surfaces for support or bearing, or a pair of functional surfaces which allows a relative movement therebetween.

However, transmission of torque can preferably be performed in this section, which can take place via several locations on the circumference, and support occurs on a substantially diametrically opposite side preferably with a form-fit engagement. In this embodiment, a bending moment about an axis perpendicular to the longitudinal axes is prevented such that the load on the material is lower and the tool can be designed in a more compact manner, i.e. with a smaller diameter. This embodiment thus constitutes a measure that allows for better guidance of the tool.

Furthermore, a tool segment with a longitudinal axis is provided, about which a receiving area is formed in the tool segment for insertion of a further tool segment. The receiving area comprises an opening, at least one functional surface that is orientated radially inwards, and an end face which is formed at least in sections by a functional surface that is orientated radially outwards, with one of the functional surfaces being provided for the transmission of torque to the other tool segment and the other functional surface being configured to allow a relative movement to the other tool segment.

This tool segment with its features has the advantages described above and, together with another tool segment, forms a tool with which a recess can be produced in the bone tissue for receiving an implant. The tool receiving area has the advantage that it can accommodate different tools. It is therefore possible, for example, to first of all prepare the recess in the bone tissue with a drill and then to carry out subsequent work with a reamer. The structural configuration of the receiving area described above thereby makes it possible for the recess in the bone tissue to have an angular course which corresponds to an angular course of the geometry of the implant to be inserted.

### BRIEF DESCRIPTION OF THE FIGURES

In the accompanying figures, to which reference is made in the following in the context of the detailed description of preferred embodiments, elements having the same function and/or design are identified by the same reference numbers.

FIG. 1 shows a schematic view of an embodiment example of the tool according to the invention, which is configured in three parts,

FIG. 2 shows a schematic sectional view of an embodiment example of the tool according to the invention, which illustrates the reciprocal bearing of two tool segments, and

FIG. 3 shows a schematic, three-dimensional side view of an embodiment example of the tool according to the invention to illustrate the kinematics.

### DETAILED DESCRIPTION OF PREFERRED EMBODIMENTS

In the following description, the term “distal” refers to the side of a component which lies at the front in the direction of advancement of the tool. Accordingly, the term “proximal” refers to the side of a component which lies at the rear in the direction of advancement of the tool.

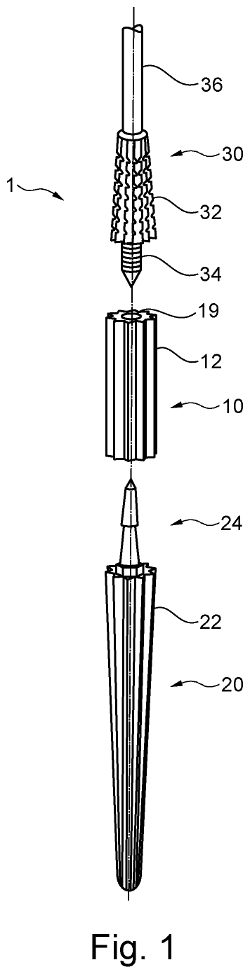

FIG. 1 shows an embodiment example of a tool 1 according to the invention, with which a recess can be produced in a long bone. The tool 1 comprises a first tool segment 10, a second tool segment 20 and a third tool segment 30.

The second tool segment 20 comprises cutting elements 22 with which a recess or hole can be produced in a bone, in particular a long bone. The tool 1, configured as a reamer, can equally be provided to widen an already existing or prepared opening in the bone tissue. The tool 1 is therefore, as already described above, provided to prepare a recess, which is adapted with respect to its geometry and its dimensions to an implant to be implanted.

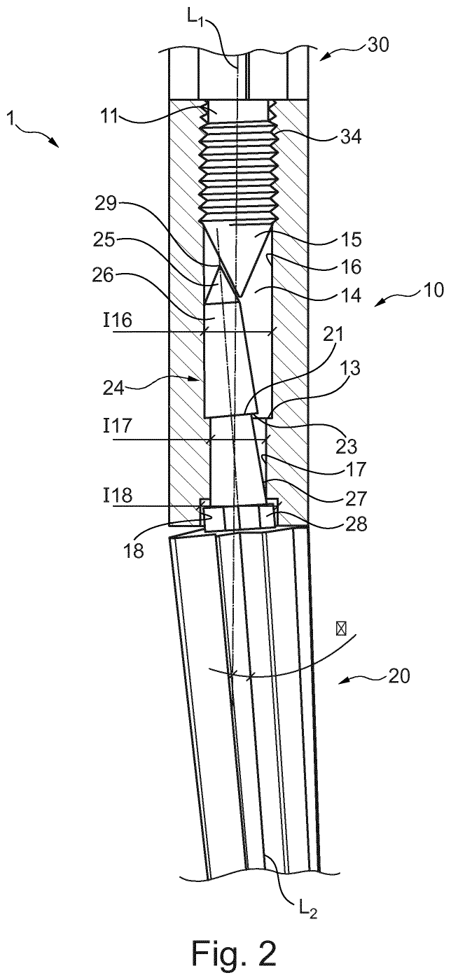

The elongated tool segment 20, which is located at the front in the direction of advancement, may have a conical shape, as shown in FIG. 2. It is also possible to configure the tool 1 to produce a cylindrical opening, as is the case for the first tool segment 10. Cutting elements 22 are provided on the circumference of the second tool segment 20 to generate the recess. By means hereof, surrounding bone tissue can be removed by rotation of the second tool segment 20 around its longitudinal axis.

Furthermore, the second tool segment 20 comprises an insertion section 24 for connection to the first tool segment and for transmitting a rotation between the two tool segments. When viewed along the longitudinal axis, the insertion section 24 is located at the rear end of the second tool segment 20 in the direction of advancement.

The insertion section 24 can be inserted into a receiving area 14, not shown, of the first tool segment 10. As is described in more detail below, the first tool segment 10 and the second tool segment 20 are thereby arranged at an angle to one another.

In the shown embodiment of the tool 1, the first tool segment 10 can also comprise cutting elements 12 arranged on the circumference in the longitudinal direction of the tool segment 10 for forming a recess or opening in the bone tissue. As is apparent in FIG. 1 in view of the circumferential surface formed by the cutting elements 12, the tool segment 10 is provided for forming a cylindrical hole. An embodiment that is tapered at least in sections can, of course, also be provided for the first tool segment 10. It is furthermore possible to only provide cutting elements 12 in sections or to provide no cutting elements 12 at all on the first tool segment 10.

The receiving area 14 (see FIG. 2), into which the second tool segment 20 can be inserted, is provided at the side located at the front in the direction of advancement of the first tool segment 10. A receiving area for a third tool segment 30 is provided on the opposite side of the first tool segment 10 in the longitudinal direction.

In the present embodiment example, the third tool segment 30 is detachably connected to a thread 19, preferably a threaded hole, of the first tool segment 10 via a projection 11 comprising a thread 34. Alternatively, other connection techniques known to the person skilled in the art for rotary tools may also be used. It is furthermore possible to configure the first tool segment 10 and the third tool segment 30 as one piece.

The third tool segment 30 can also comprise cutting elements 32, as shown in the embodiment example of FIG. 1.

Located at the distal end of the elongated third tool segment 30 is the aforementioned thread 34 for connection to the first tool segment 10. A clamping element 36 is provided at the opposite or proximal end of the third tool segment 30, which is configured to be cylindrical in the present embodiment and can be clamped, for example, in a drill. A torque introduced via the connecting element 36 can be transmitted from the third tool segment 30 to the first tool segment 10 and, in turn, from this segment to the second tool segment 20.

This modular design of the tool 1 has the advantage that it can be adapted very well to the implant to be implanted or to the opening to be created herefor in the bone tissue.

FIG. 2 shows a sectional view through the first tool segment 10, which is connected at its proximal end to a third tool segment 30 and at its distal end to a second tool segment 20. In the illustrated embodiment of the first tool segment 10, it comprises a through-hole. This through-hole is provided at its proximal end with a thread 19 for receiving a threaded portion 34 on a distal projection of the third tool segment 30. The free space remaining in the through-hole after screwing in the threaded portion 34 forms the receiving area 14 for the insertion section 24 of the second tool segment 20.

Viewed from the proximal to the distal end, the receiving area 14 comprises a second functional surface 16 that is orientated radially inwards, an annular holding surface 13, a third functional surface 17 that is orientated radially inwards and a fourth functional surface 18 that is orientated radially inwards. The functional surfaces are preferably cylindrical, as shown, and are more preferably circular cylindrical. As is described below within the context of modifications of the present embodiment, fewer functional surfaces may also be provided.

The second functional surface 16 that is orientated radially inwards has an internal dimension I16 which is greater than the internal dimension I17 of the third functional surface 17 that is orientated radially inwards. Due to the resulting extension of the receiving area 14 in the proximal direction, a shoulder is formed, which forms an annular holding surface 13 in the receiving area 14 of the second tool segment 20. The annular holding surface 13 is preferably formed perpendicular to the longitudinal axis L1 of the first tool segment 10. The fourth functional surface that is orientated radially inwards has an internal dimension 118, which is in turn greater than the diameter I17.

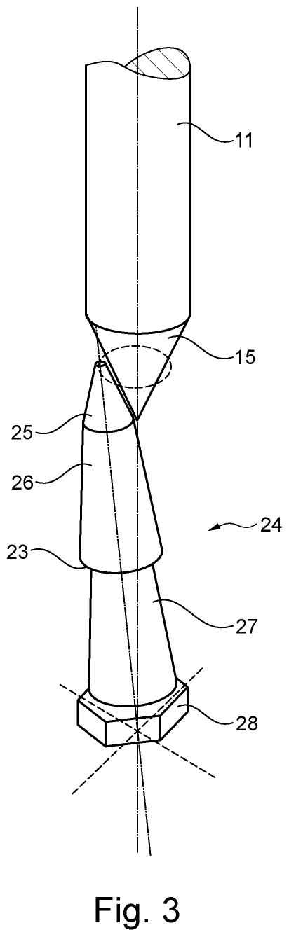

In the embodiment illustrated in FIG. 2, the functional surfaces 16 and 17 that are orientated radially inwards are circular cylindrical. The internal dimension I16 of the second functional surface 16 and the internal dimension I17 of the third functional surface 17 are consequently internal diameters. By contrast, the fourth functional surface 18 of the first tool segment 10 that is orientated radially inwards is formed with a geometry that allows engagement with a corresponding functional surface 28 of the insertion section for transmission of a torque. It is a hexagon in the present embodiment example in FIGS. 2 and 3. It is to be understood that any other polygon or geometry may be used, provided that it allows torque to be transmitted. This applies accordingly to the fourth functional surface 28 of the second tool segment 20 that is orientated radially outwards.

Furthermore, located in the receiving area 14 of the first tool segment 10 is a first functional surface 15 that is orientated radially outwards, which is formed in the present embodiment by the projection 11 of the third tool segment 30 that is provided with a thread 34. It is alternatively possible to configure the first functional surface 15 that is orientated radially outwards as part of the first tool segment 10. In the embodiment illustrated in FIG. 2, the functional surface 15 is formed as a conical surface.

When viewed from the proximal to distal end, the insertion portion 24 of the second tool segment 20 inserted into the receiving area 14 comprises a first functional surface 25 that is orientated radially outwards, a second functional surface 26 that is orientated radially outwards, a third functional surface 27 that is orientated radially outwards and, as already mentioned above, a fourth functional surface 28 that is orientated radially outwards.

The functional surfaces 25, 26 and 27 taper in the illustrated embodiment from the distal end towards the proximal end point 29 of the insertion section 24. The first functional surface 25 of the second tool segment 20 that is orientated radially outwards is conical just like the first functional surface 15 of the first tool segment 10 that is orientated radially outwards. At least one of the functional surfaces 15 and 25 may likewise have the shape of a truncated cone.

The second functional surface 26 that is orientated radially outwards connects distally to functional surface 25 that is orientated radially outwards, and has the shape of a truncated cone. The functional surface 27, which also has the shape of a truncated cone, adjoins the functional surface 26. It has a smaller diameter at its proximal end than the distal base 21 of the truncated cone of the functional surface 26, which is also located at this proximal end. As a result, a shoulder is formed by the functional surface 26 and the functional surface 27. The shoulder surface serves as an annular support surface 23, which, as described below, interacts with the holding surface 13 of the first tool segment.

As described above, the tool 1 comprises, for the transmission of torque, at least one pair of functional surfaces that allows a relative movement therebetween, and at least one pair of functional surfaces that is engaged with a form fit or friction fit.

In the tool, the engaged pair of functional surfaces forms the torque transmitting device for transmitting a torque between the tool segments. With the aid of this torque, a cutting movement is performed by at least one of the tool segments. The pair of functional surfaces that allows a relative movement, i.e. slipping, in its contact region is on the other hand provided for support and guidance between the first and second tool segments, and thus transfers the forces in the longitudinal direction between the tool segments.

In the embodiment example illustrated in FIG. 2, more pairs of functional surfaces are, however, used in order to achieve as defined a sequence of movement as possible between the first tool segment 10 and the second tool segment 20.

The pair of functional surfaces which allows a relative movement therebetween is formed by the functional surfaces 15 and 25. As described above, the engaged pair of functional surfaces is formed by the functional surfaces 18 and 28. The functional surfaces 18 and 28 are thereby engaged with a form fit, which in general ensures a better transmission of torque than a friction-fit engagement. Furthermore, the effective or pitch circle diameter of the engaged functional surfaces 18 and 28 is approximately at the height of the intersection of the longitudinal axis L1 of the first tool segment 10 and the longitudinal axis L2 of the second tool segment 20.

The effective circle diameter or pitch circle diameter of a functional surface is the diameter on which lie the contact points where the perimeters of two interacting functional surfaces come into contact. As in gear technology, a gear ratio can be determined by the ratio between the effective circle diameters of two gear elements, for example two gearwheels. Since the effective circle diameters of the functional surfaces 18 and 28 are at the height of the intersection between the longitudinal axes L1 and L2 and since the longitudinal axis L1 forms the central point of the effective circle diameter of the functional surface 18 and the longitudinal axis L2 forms the central point of the effective circle diameter of the functional surface 28, the two effective circle diameters are approximately the same size, and thus the gear ratio is approximately 1:1.

Furthermore, a pair of functional surfaces is provided in the embodiment shown in FIG. 2, which supports the insertion section 24 in the receiving area 14. This pair consists of the second functional surface 16 of the first tool segment 10 which is orientated radially inwards and the second functional surface 26 of the second tool segment 20 which is orientated radially outwards. Alternatively or additionally, the pair of functional surfaces comprising the third functional surface 17 of the first tool segment 10 which is orientated radially inwards and the third functional surface 27 of the second tool segment 20 which is orientated radially outwards may be provided as a supporting functional surface pair.

The surfaces of the supporting and guiding functional surface pair roll against one another. Consequently, they allow substantially no relative movement between them and are also not provided for transmission of torque.

The interaction between the three pairs of functional surfaces is complemented in the present embodiment by supporting the support surface 23 on the holding surface 13 in the receiving area 14. The support of the support surface 23 on the holding surface 13 in particular ensures a defined contact between the functional surfaces 15 and 25 and the functional surfaces 26 and 16. At the same time, the insertion section 24 of the second tool segment 20 is reliably held in the receiving area 14 of the first tool segment 10.

So that the insertion section 24 can be inserted into the receiving area 14 in such a manner that the support surface 23 of the second tool segment 20 can reach behind the holding surface 13 of the first tool segment 10 in the proximal direction, a section of the insertion section 24 and/or the functional surface 15 that is orientated radially outwards is preferably configured to be elastically flexible. Alternatively or additionally, insertion of the insertion section 24 can take place in that the tool segment 30 is only completely screwed into the first tool segment 10 once the insertion section 24 of the second tool segment 20 has been inserted through the distal opening of the receiving area 14.

In the simplest case, however, the insertion section 24 and the receiving area 14 are dimensioned such that insertion of the insertion section 24 is possible without an elastic flexibility that is specifically provided therefor or without locking by the projection 11 of the third tool segment. The holding surface 13 and the support surface 23 hereby substantially prevent the second tool segment 20 from inadvertently falling out of the first tool segment 10, but not, however, with the same degree of reliability as the two previously described embodiments. On the other hand, it is particularly easy to exchange the first tool segment 10 in this embodiment.

Driving of the tool segment 20 takes place via the clamping element 36 shown in FIG. 1. The rotation introduced via this element is transmitted directly to the first tool segment 10. The transmission of torque from the first tool segment 10 to the second tool segment 20 then occurs, as described above, at the height of the pair of functional surfaces 18, 28 via the insertion section 24 that is inserted into the receiving area 14. However, the kinematic sequence of motion is largely determined by the pair of functional surfaces 15, 25 and the pair of functional surfaces 16, 26 and/or the pair of functional surfaces 17, 27.

As described above, the path of the tool 1 through the bone tissue results from a pilot hole, a bone density distribution in the bone tissue and/or a medullary cavity of the bone to be treated. The tool segment 20 thereby generally takes the path of least resistance. This path of least resistance thus guides the tool segment 20.

If initially only the tool segment 20 is guided, for example if only this segment is located in the bone tissue, this results in two movement possibilities that have already been described above between the first tool segment 10 and the second tool segment 20. On the one hand, the insertion section 24 with the longitudinal axis L2 can move on a double-cone circular path relatively about the longitudinal axis L1 of the first tool segment 10. In this state, a rotation can at the same time be transmitted from the first tool segment 10 to the second tool segment 20.

The movement of the insertion section 24 on a double-cone circular path relatively about the longitudinal axis L1 of the first tool segment 10 results, with reference to FIG. 2, owing to the circular tool path predetermined by the functional surface 15 and the functional surface 16. As an alternative to a circular tool path, other tool paths are also conceivable, such as, for example, a tool path which, due to a correspondingly formed geometry of the functional surface 16 and the functional surface 15, extends in an undulating manner about the longitudinal axis L1. In such an embodiment, the angle α between the longitudinal axes L1 and L2 changes depending on the angular position of the longitudinal axis L1 to the longitudinal axis L2.

If the first tool segment 10 shown in FIG. 2 is guided, for example by a surgeon's hand, the first tool segment 10 and the second tool segment 20 remain in the same position relative to a global coordinate system, such as the position shown in FIG. 2 or 3. The angle α of the tool segments 10 and to one another results from the angle between the two longitudinal axes L1 and L2, which is in turn determined by the taper angle of the functional surfaces 15 and 25.

Some possible modification possibilities of the embodiment shown in FIGS. 2 and 3 will be described below.

In the most simply constructed embodiment, transmission of rotation and support occurs between functional surfaces 15, 25 and 16, i.e. on the one hand at a position at which the functional surfaces 15 and 25 contact one another, and on the other hand at a position that is approximately diametrically opposite to this position, at which the functional surfaces 25 and 16 contact one another.

In this embodiment, the functional surfaces must be accordingly geometrically adjusted. For example, the second functional surface 16 of the first tool segment 10 that is orientated radially inwards may taper in the proximal direction. This embodiment also has a pair of functional surfaces that allows a relative movement therebetween and a pair of functional surfaces via which the transmission of torque is made possible. However, the pairs of functional surfaces overlap in this embodiment.

If the pair of functional surfaces that allows a relative movement therebetween is the pair of functional surfaces consisting of the first functional surface 15 that is orientated radially outwards relative to the longitudinal axis L1 and the first functional surface 25 that is orientated radially outwards relative to the longitudinal axis L2, then the pair of functional surfaces that enables transmission of a rotation or torque is between the second functional surface 16 and the first functional surface 25. In this case, the first tool segment 10 and the second tool segment 20 rotate in the same direction.

Depending on the choice of the respective effective circle diameters of the functional surface 25 and the functional surface 16, it is possible for the second tool segment 20 to rotate at a speed that differs from that of the tool segment 10 by the ratio between the effective circle diameters. In addition to the simple construction, an increase in the rotational speed of the first tool segment 10 relative to the second tool segment 20 is thus possible in this embodiment.

In a further alternative embodiment, torque transmission can occur between functional surfaces 15 and 25 whilst relative movement is allowed between functional surfaces 16 and 25. In this case as well, the rotational speed between the two tool segments 10 and 20 depends on the ratio of the effective circle diameters of the two functional surfaces 15 and 25. In this embodiment, however, the second tool segment 20 additionally rotates in the opposite direction in relation to the first tool segment 10. In this embodiment, there is consequently a reversal of the direction of rotation in addition to a gear ratio.

In order to achieve a more stable kinematic behavior of the tool segments 10 and 20 relative to one another, two separate pairs of functional surfaces can be provided. Applied to FIG. 2, the first pair of functional surfaces could be formed from functional surfaces 15 and 25, and the second pair of functional surfaces could be formed from functional surfaces 16 and 26. Alternatively, one of the remaining pairs of functional surfaces 17 and 27 or 18 and 28 could also be used.

In this embodiment, the relative movement occurs, for example, between the pair of functional surfaces 15, 25, whilst transmission of the rotational movement occurs by means of an engagement between the pair of functional surfaces 16, 26. As described above, the tool segment 10 and the tool segment 20 rotate in the same direction in this case, with the angular velocities of the tool segment 10 relative to the tool segment 20 depending on the ratio of the effective circle diameters of the functional surface 26 relative to the functional surface 16.

In the reverse case, i.e. an engagement between the functional surfaces 15 and 25 and a relative movement between the functional surfaces 16 and 26, the direction of rotation between the tool segments 10 and 20 is opposite to that of the preceding embodiment. The ratio of the effective circle diameters to one another again applies for the rotational speed.

Also in the case of the described modifications, a holding surface 13 is preferably provided on the first tool segment and a support surface 23 is preferably provided on the second tool segment.

Furthermore, in the modifications, there is a difference as regards the rotational speeds of the two tool segments since, differing from the embodiment example in FIG. 2, the torque transmission does not lie at the height of the intersection between the longitudinal axis L1 of the first tool segment 10 and the longitudinal axis L2 of the second tool segment 20. Consequently, the effective circle diameters of the insertion section 24 and the receiving area 14 or the third tool section 30 differ from one another.

Furthermore, more than two tool segments can also be at an angle to one another according to the invention. For example, the third tool segment can also be at an angle to the first tool segment and thus form a recess with two angles that is intended for an implant.

The present tool is intended in particular for implants implanted in long bones. These include in particular hip implants since, in this case, the property of the tool that it is able to prepare a hole provided with an angle is of particular advantage in view of the curved course of the natural neck of the femur.

### LIST OF REFERENCE NUMBERS

1 Tool
10 First tool segment
11 Projection in the receiving area
12 Cutting element
13 Holding surface in the receiving area
14 Receiving area
15 (First) functional surface of the first tool segment that is orientated radially outwards
16 (Second) functional surface of the first tool segment that is orientated radially inwards
17 (Third) functional surface of the first tool segment that is orientated radially inwards
18 (Fourth) functional surface of the first tool segment that is orientated radially inwards
19 Threaded hole
20 Second tool segment
21 Distal base of the truncated cone formed by the functional surface 26
22 Cutting element
23 Supporting surface
24 Insertion section
25 (First) functional surface of the second tool segment that is orientated radially outwards
26 (Second) functional surface of the second tool segment that is orientated radially outwards
27 (Third) functional surface of the second tool segment that is orientated radially outwards
28 (Fourth) functional surface of the second tool segment that is orientated radially outwards
29 End point of the insertion section
30 Third tool segment
32 Cutting element
34 Threaded section
36 Clamping element
I16 Interior dimension of the second functional surface of the first tool segment
I17 Interior dimension of the third functional surface of the first tool segment
I18 Interior dimension of the fourth functional surface of the first tool segment
L1 First longitudinal axis
L2 Second longitudinal axis
α Angle between the longitudinal axes L1 and L2

1-12. (canceled)

13. A tool (1) for producing a recess in bone tissue for an implant, said tool comprising:
a first elongated tool segment (10) with a first longitudinal axis (L1) and a receiving area (14), said receiving area having, about the first longitudinal axis, a first functional surface (15) that is orientated substantially radially outwards and a second functional surface (16, 17, 18) that is orientated radially inwards,
a second elongated tool segment (20) with a second longitudinal axis (L2) and an insertion section (24) that can be inserted into the receiving area of the first tool segment, said insertion section having, about the second longitudinal axis, at least one functional surface (25, 26, 27, 28) that is orientated radially outwards and interacts with the first functional surface (15) of the first tool segment that is orientated radially outwards, and one of the at least one functional surfaces (25, 26, 27, 28) of the insertion section interacting with the second functional surface (16, 17, 18) of the first tool segment,
wherein the first longitudinal axis and the second longitudinal axis intersect, one of the interacting pairs of functional surfaces (15, 16, 17, 18, 25, 26, 27, 28) is engaged with one another for transmitting torque, and the other of the interacting pairs of functional surfaces (15, 16, 17, 18, 25, 26, 27, 28) allows a relative movement therebetween.

14. A tool (1) according to claim 13, wherein the first functional surface (15) of the first tool segment (10) is formed by a projection (11) in the receiving area (14), said projection preferably being conical or cylindrical.

15. A tool (1) according to claim 13, wherein the engaged pair of functional surfaces (15, 16, 17, 18, 25, 26, 27, 28) for the transmission of torque is engaged with a form-fit.

16. A tool (1) according to claim 14, wherein the engaged pair of functional surfaces (15, 16, 17, 18, 25, 26, 27, 28) for the transmission of torque is engaged with a form-fit.

17. A tool (1) according to claim 13, wherein at least the cross-sections of the functional surfaces (15, 16, 17, 18, 25, 26, 27, 28) of at least one pair of functional surfaces perpendicular to the respective longitudinal axis (L1, L2) have a substantially circular circumference.

18. A tool (1) according to claim 14, wherein at least the cross-sections of the functional surfaces (15, 16, 17, 18, 25, 26, 27, 28) of at least one pair of functional surfaces perpendicular to the respective longitudinal axis (L1, L2) have a substantially circular circumference.

19. A tool according to claim 13, wherein the first tool segment (10) and the second tool segment (20) comprise at least two separate functional surface pairs (15, 16, 17, 18, 25, 26, 27, 28) interacting therebetween.

20. A tool according to claim 14, wherein the first tool segment (10) and the second tool segment (20) comprise at least two separate functional surface pairs (15, 16, 17, 18, 25, 26, 27, 28) interacting therebetween.

21. A tool according to claim 13, wherein the first tool segment (10) and the second tool segment (20) comprise at least three separate functional surface pairs (15, 16, 17, 18, 25, 26, 27, 28) interacting therebetween, wherein the first pair of functional surfaces (15, 25) permits a relative movement therebetween, the second pair of functional surfaces (16, 26) supports the insertion section (24) in the receiving area (14), and the torque can be transmitted between the first and the second tool segments by the third pair of functional surfaces (18, 28).

22. A tool according to claim 14, wherein the first tool segment (10) and the second tool segment (20) comprise at least three separate functional surface pairs (15, 16, 17, 18, 25, 26, 27, 28) interacting therebetween, wherein the first pair of functional surfaces (15, 25) permits a relative movement therebetween, the second pair of functional surfaces (16, 26) supports the insertion section (24) in the receiving area (14), and the torque can be transmitted between the first and the second tool segments by the third pair of functional surfaces (18, 28).

23. A tool according to claim 13, wherein the functional surface (15) of the first tool segment (10) that is orientated radially outwards is exchangeable and preferably part of a third tool segment (30).

24. A tool according to claim 14, wherein the functional surface (15) of the first tool segment (10) that is orientated radially outwards is exchangeable and preferably part of a third tool segment (30).

25. A tool according to claim 13, wherein at least one of the tool segments (10, 20, 30) comprises at least one cutting element (12, 22, 32) on an outward facing circumferential surface.

26. A tool according to claim 14, wherein at least one of the tool segments (10, 20, 30) comprises at least one cutting element (12, 22, 32) on an outward facing circumferential surface.

27. A tool according to claim 13, wherein the receiving area (14) of the first tool segment (10) comprises a holding surface (13) which is formed by a cross-sectional extension within the receiving area and on which a corresponding cross-sectional extension of the insertion section (24) is supported in the inserted state such that the second tool segment is held in the first tool segment.

28. A tool according to claim 14, wherein the receiving area (14) of the first tool segment (10) comprises a holding surface (13) which is formed by a cross-sectional extension within the receiving area and on which a corresponding cross-sectional extension of the insertion section (24) is supported in the inserted state such that the second tool segment is held in the first tool segment.

29. A tool (1) according to claim 13, wherein a pair of functional surfaces is arranged at the height of the intersection of the first longitudinal axis (L1) and the second longitudinal axis (L2), said pair comprising a functional surface (26, 27, 28) of the insertion section (24) that is orientated radially outwards and a functional surface (16, 17, 18) of the receiving area (14) that is orientated radially inwards, and the pair of functional surfaces is preferably suitable for transmitting torque.

30. A tool (1) according to claim 14, wherein a pair of functional surfaces is arranged at the height of the intersection of the first longitudinal axis (L1) and the second longitudinal axis (L2), said pair comprising a functional surface (26, 27, 28) of the insertion section (24) that is orientated radially outwards and a functional surface (16, 17, 18) of the receiving area (14) that is orientated radially inwards, and the pair of functional surfaces is preferably suitable for transmitting torque.

31. A tool segment (10) with a longitudinal axis (L1), about which a receiving area for insertion of a further tool segment (20) is formed, said receiving area comprising an opening, a functional surface (16, 17, 18) that is orientated radially inwards, and an end face formed at least in sections by a functional surface (15) that is orientated radially outwards, wherein one of the functional surfaces (15, 16, 17, 18) is provided for the transmission of torque to the other tool segment and the other functional surface (15, 16, 17, 18) is configured to allow a relative movement to the other tool segment.

32. A set consisting of a tool according to claim 13 and an implant, in particular an endoprosthesis, which can be inserted into a recess that can be generated by the tool in the bone tissue of a long bone.
